(19) United States
(12) Patent Application Publication
(10) Pub. No.: US 2024/0347142 A1
(43) Pub. Date: Oct. 17, 2024

(54) METHOD AND ELECTRONIC DEVICE FOR LIGAND GENERATION

(71) Applicants: Lemon Inc., Grand Cayman (KY); Beijing Youzhuju Network Technology Co., Ltd., Beijing (CN)

(72) Inventors: Yuwei YANG, Los Angeles, CA (US); Xiangxin ZHOU, Beijing (CN); Yu BAO, Beijing (CN); Jiaqi GUAN, Los Angeles, CA (US)

(21) Appl. No.: 18/632,639

(22) Filed: Apr. 11, 2024

(30) Foreign Application Priority Data
Apr. 13, 2023 (CN) 202310395913.7

(51) Int. Cl. G16C 20/50; G16C 20/30

(52) U.S. Cl. CPC G16C 20/50; G16C 20/30

(57) ABSTRACT

Embodiments of the present disclosure relate to a method and an electronic device for ligand generation. The method comprises: obtaining a trained ligand generation model, wherein the trained ligand generation model is generated based on decomposition of a ligand by modeling of atom positions, atom types, and chemical bonds; and obtaining, based on a target protein, a target ligand molecule corresponding to the target protein by using the trained ligand generation model. According to the method, the ligand generation model in the embodiments of the present disclosure considers the decomposition of the ligand and also models the chemical bonds, so that it can generate the target ligand molecule with higher affinity.

### CROSS-REFERENCE TO RELATED APPLICATION(S)

This application claims priority to Chinese Application No. 202310395913.7 filed Apr. 13, 2023, the disclosure of which is incorporated herein by reference in its entity.

### FIELD

The present disclosure generally relates to the field of computers and the field of biological information, and more specifically, to a method and an electronic device for ligand generation.

### BACKGROUND

Interactions between biomolecules are an important foundation for achieving the biological activities of the biomolecules. For example, the human body can produce antibody proteins that bind to invading viruses to suppress diseases. In biopharmaceutical studies, physical and chemical mechanisms of intermolecular interactions can be understood by analyzing known molecules that bind to each other, thereby helping design a novel drug molecule that can bind to specific targets. For example, a ligand molecule that binds to a target protein can be determined, and then the drug can be designed based on the ligand molecule. Therefore, how to efficiently determine the ligand molecule that can bind to the target protein is one of the current problems that need to be solved.

### SUMMARY

According to exemplary embodiments of the present disclosure, a method for ligand generation is provided. A target ligand molecule corresponding to a target protein is obtained based on a trained ligand generation model, and then the target ligand molecule can be applied for a drug design.

A first aspect of the embodiments of the present disclosure provides a method for ligand generation. The method includes: obtaining a trained ligand generation model, wherein the trained ligand generation model is generated based on decomposition of a ligand by modeling of atom positions, atom types, and chemical bonds; and obtaining, based on a target protein, a target ligand molecule corresponding to the target protein by using the trained ligand generation model.

A second aspect of the embodiments of the present disclosure provides an electronic device, including: at least one processing unit; and at least one memory, wherein the at least one memory is coupled to the at least one processing unit and stores instructions executed by the at least one processing unit, and the instructions, when executed by the at least one processing unit, cause the electronic device to perform the method described according to the first aspect of the present disclosure.

A third aspect of the embodiments of the present disclosure provides a computer-readable storage medium. The computer-readable storage medium has machine-executable instructions stored thereon. The machine-executable instructions, when executed by a device, cause the device to perform the method described according to the first aspect of the present disclosure.

A fourth aspect of the embodiments of the present disclosure provides a computer program product, including computer-executable instructions. The computer-executable instructions, when executed by a processor, implement the method described according to the first aspect of the present disclosure.

A fifth aspect of the embodiments of the present disclosure provides an electronic device, including: a processing circuit, configured to perform the method described according to the first aspect of the present disclosure.

The provision of the summary section is intended to introduce a series of concepts in a simplified form, which will be further described in specific implementations below. The summary section is not intended to identify the key or necessary features of the present disclosure, nor is it intended to limit the scope of the present disclosure. Other features of the present disclosure will be easily understood through the following description.

### BRIEF DESCRIPTION OF THE DRAWINGS

The above and other features, advantages, and aspects of the various embodiments of the present disclosure will become more apparent in combination with the accompanying drawings and referring to the following detailed descriptions. In the accompanying drawings, identical or similar reference numerals represent identical or similar elements.

FIG. 1 shows a schematic diagram of decomposition of a ligand molecule;

FIG. 2 shows a flowchart of an exemplary training process according to some embodiments of the present disclosure;

FIG. 3 shows a flowchart of an exemplary use process according to some embodiments of the present disclosure;

FIG. 4 shows a schematic diagram of generating a target ligand molecule according to some embodiments of the present disclosure;

FIG. 5 shows a block diagram of an exemplary apparatus according to some embodiments of the present disclosure; and

FIG. 6 shows a block diagram of an exemplary device configured to implement an embodiment of the present disclosure.

### DETAILED DESCRIPTION OF EMBODIMENTS

The embodiments of the present disclosure will be described in detail below with reference to the accompanying drawings. Although the accompanying drawings show some embodiments of the present disclosure, it should be understood that the present disclosure can be implemented in various forms, and should not be explained as being limited to the embodiments stated herein. Rather, these embodiments are provided for understanding the present disclosure more thoroughly and completely. It should be understood that the accompanying drawings and embodiments of the present disclosure are only used for illustration, but are not intended to limit the protection scope of the present disclosure.

In biopharmaceutical studies, according to data types that the methods relay on, drug design methods can include a ligand-based drug design method and a structure-based drug design method.

The ligand-based drug design method collects a large amount of existing ligand information for a specific target, and can explore, based on the ligand structure, a rule of the molecular that binds to the specific target, thereby designing or optimizing the ligand structure.

The ligand-based drug design method does not rely on structural data of a target and only requires ligand information, so it can only be applied to targets that already have a large amount of ligand information. A ligand-based drug design model can be constructed based on deep learning, such as a one-dimensional simplified molecular input line entry system (SMILES) or a two-dimensional molecular graph that can be used to generate molecules.

The structure-based drug design method can design, based on a 3D structure of a target, a 3D ligand structure that can closely interact with the 3D structure of the target. Specifically, relative positions between atoms of the ligand and the target can be used for estimating interaction forces or the suitability between the ligand and the target, thereby the 3D ligand structure can be designed and optimized. The structure-based drug design method considers the 3D structures of both the target and the ligand, and achieves a drug molecule design by satisfying an appropriate interaction force purpose, thus it can apply to any target with a 3D structure. A structure-based drug design model can be constructed based on deep learning. For example, a target-ligand 3D structure may be used as training data, and this structure-based drug design model aims to generate a 3D ligand structure in a binding pocket of the target.

The structure-based drug design model based on deep learning typically regards the ligand molecule as a whole, and predicts types and positions (which are represented as coordinates for example) of ligand atoms using a generation technology such as a conditional variational autoencoder, autoregression, and a diffusion model, thereby completing the design task of the 3D ligand molecule. One of the existing structure-based drug design models is a 3D drug design model based on a diffusion model, which uses the diffusion model to model a distribution of atom types and atom coordinates of atoms in the ligand molecule. The 3D drug design model based on the diffusion model has a relatively low risk of generating an accumulative error, utilize the interaction forces between the atoms better, and can generate a 3D ligand structure with the higher affinity to the target.

However, the current schemes still need a further improvement. For example, the affinity between the generated ligand structure and the target needs to be further improved. For example, the 3D drug design model based on the diffusion model assumes that a prior distribution of the ligand molecule follows a standard Gaussian distribution, so that the affinity between the generated ligand structure and the target is not enough. Due to the lack of a direct constraint on a position relationship between the ligand atoms and the target, the sampled ligand molecule may be too close to the target or even collide with the target, thus the affinity may be not sufficient due to the caused mutual repulsion.

In view of this, the embodiments of the present disclosure provide a scheme for ligand generation to address the aforementioned issues and other potential issues. Specifically, a target ligand molecule corresponding to a target protein can be obtained based on a trained ligand generation model, and then the target ligand molecule can be applied to the drug design.

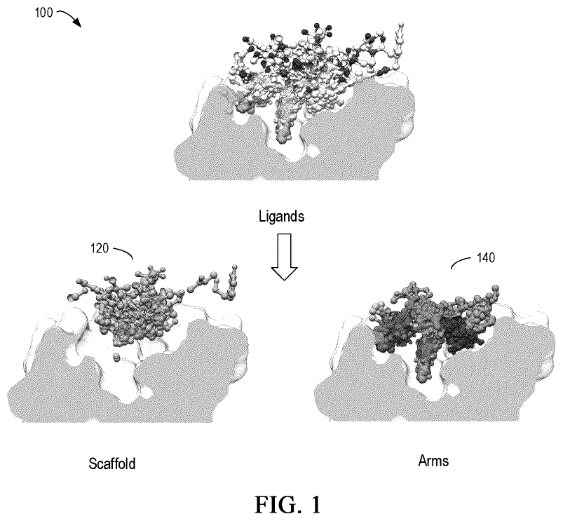

As considered in the traditional drug design, a ligand molecule can be divided into a scaffold structure and a plurality of functional groups connected to the scaffold. Exemplarily, the scaffold structure can also be referred to as a scaffold, and the functional groups can also be referred to as arms. According to a relative position of the binding between the ligand and the target, the functional groups are usually in close contact with a surface of the target to play a role of improving the activity, and the scaffold supports the functional groups and places the functional groups to appropriate binding areas. FIG. 1 shows a schematic diagram of decomposition 100 of a ligand molecule. As shown in FIG. 1, the ligand molecule is divided into a scaffold 120 and arms 140.

In the embodiments of the present disclosure, a ligand generation model based on a diffusion model is provided. This model can simultaneously generate the atom types, atom positions, and chemical bonds of the ligand molecule, thereby improving the affinity between the ligand and the target.

In the embodiments of the present disclosure, the term “target” can also be referred to as a target protein, a protein target, a receptor protein, a receptor, a protein, and the like. The term “ligand” can also be referred to as a ligand molecule, a ligand small molecule, and the like.

In the embodiments of the present disclosure, a ligand generation model can be used to generate a target ligand molecule corresponding to a target protein. This ligand generation model may be a ligand generation model based on the diffusion model. The ligand generation model based on the diffusion model may be generated based on decomposition of atoms in the ligand. Therefore, it can also be referred to as a decomposable diffusion (DecompDiff) model in the present disclosure.

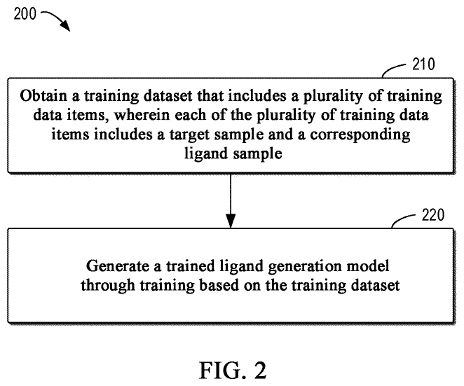

FIG. 2 shows a flowchart of an exemplary training process 200 according to some embodiments of the present disclosure. At block 210, a training dataset that includes a plurality of training data items is obtained, wherein each of the plurality of training data items includes a target sample and a corresponding ligand sample. At block 220, a trained ligand generation model is generated through training based on the training dataset.

The training dataset can be referred to as a training set or a dataset, which includes a plurality of data items, and each of which includes a target sample and a corresponding ligand sample, i.e., a pair of a target sample and a ligand sample, also referred to as a target-ligand pair.

In some embodiments of the present disclosure, a decomposition prior distribution can be determined based on the target sample and the ligand sample; an atom neighbor graph is constructed, wherein the atom neighbor graph represents relationships between a plurality of atoms that are closely near to each other; a fully connected graph is constructed, wherein the fully connected graph represents relationships between different atoms of the ligand sample; and the trained ligand generation model is generated through training based on the decomposition prior distribution, the atom neighbor graph, and the fully connected graph.

The process of determining the decomposition prior distribution can be referred to as a data preparation stage. For example, a scaffold-arm decomposition prior can be obtained. Exemplarily, a position of a local binding pocket on the target sample can be determined; the plurality of atoms of the ligand sample can be classified into a plurality of clusters based on a binding situation between the ligand sample and the local binding pocket on the target sample; and mean matrixes and covariance matrixes of atom positions in the plurality of clusters can be further determined, thereby the decomposition prior distribution can be obtained.

Optionally, the position of the local binding pocket on the target sample can be determined using an existing target binding site prediction method. Exemplarily, the local binding pocket on the target sample is determined based on geometric information (such as a curvature), chemical information (such as an affinity), and the like. For example, the position of the local binding pocket on the target sample can be extracted using target binding site prediction software such as AlphaSpace.

Optionally, the atoms of the ligand sample can be classified through a clustering algorithm according to binding situations between all the atoms in the ligand sample and the local binding pockets on the target sample. For example, atoms that occupy the same local binding pocket can be classified to one arm atoms cluster, and atoms that do not occupy the local binding pocket can be classified to a scaffold atoms cluster. For example, the plurality of clusters obtained by the classification may include a scaffold atoms cluster and a plurality of arm atoms clusters.

Optionally, for each of the clusters, a mean matrix and a covariance matrix of the atom coordinate distribution can be obtained through a method such as maximum likelihood estimation based on the positions (such as coordinates) of the atoms in the cluster. In this way, a combination of the mean matrixes and covariance matrixes of the plurality of clusters can be understood as the decomposition prior distribution. In other words, the decomposition prior distribution of the ligand sample in a certain data item includes the mean matrix and covariance matrix for each of the plurality of clusters that is obtained by decomposition. For example, a plurality of prior distributions corresponding to the plurality of clusters include a scaffold prior distribution and a plurality of arm prior distributions. Assuming that the total number of the plurality of clusters is a positive integer K, the mean matrix of a k-th cluster among the plurality of clusters can be represented as μₖ, and the covariance matrix of the k-th cluster can be represented as Σₖ, where the value of k may be a positive integer within 1˜K.

The process of constructing the neighbor graph and the fully connected graph can be referred to as a stage of extracting data features. Exemplarily, nodes in the atom neighbor graph may represent the atoms of the target sample or the atoms of the ligand sample. Optionally, a feature of a node corresponding to an atom of the target sample in the atom neighbor graph includes at least one of the following of the atom: an atom type, an atom position, an amino acid type, an indication of whether the atom is located on a main chain, and an indication of one or more arms with the atom being located within a predetermined range therein. Optionally, a feature of a node corresponding to an atom of the ligand sample in the atom neighbor graph includes at least one of the following of the atom: an atom type, an atom position, and an indication of an arm or a scaffold that the atom belongs to. Optionally, a feature of an edge in the atom neighbor graph includes a distance between two atoms connected by the edge and a type of the edge, where the type of the edge includes any of the following: a target-ligand connecting edge, a target-target connecting edge, a ligand-target connecting edge, or a ligand-ligand connecting edge. Exemplarily, a feature of a node of the fully connected graph includes at least one of the following of an atom: an atom type, an atom position, an indication of whether the atom belong to a scaffold, or an indication of whether the atom belongs to an arm; and a feature of an edge in the fully connected graph includes a chemical bond type and an indication whether two atoms connected by the edge belong to the same prior distribution.

Optionally, it can be determined that the feature of the atom in the target sample, which is denoted as [symbol] , may represent one or more of the following: the atom type (such as one of H, C, N, O, S, Se, F, P, and Cl), the atom position (which is represented as coordinates for example), the amino acid type (such as one of 20 types), the indication of whether the atom is located on the main chain, the indication of one or more arms with the atom being located within the predetermined range (such as 10 Å) therein, and the like.

Optionally, it can be determined that the feature of the atom in the ligand sample, which is denoted as [symbol] , may represent one or more of the following: the atom type (such as H, C, N, O, S, Se, F, P, Cl, etc.), the atom position (which is represented as coordinates for example), the indication of whether the atom belongs to an arm or a scaffold, or the indication of the arm or scaffold the atom belongs to. For example, if the distance between an atom and a center (μₖ) of any arm prior distribution is less than a threshold (such as 10 Å), it can be determined that the atom belongs to the arm; otherwise, it can be determined that the atom belongs to the scaffold.

The neighbor graph and the fully connected graph can be generated based on the features [symbol] of the atoms in the target sample and the features [symbol] of the atoms in the ligand sample. Optionally, a k-neighbor graph can be constructed on the atoms in the target sample and the atoms in the ligand sample, which is represented as [symbol] _{K}. For example, taking an atom (a target atom or a ligand atom) as a center, k atoms nearest to the atom of the center are determined to construct the k-neighbor graph. In the graph [symbol] _{K}, nodes represent atoms, and node features are the aforementioned atom features ( [symbol] , [symbol] ). The features of an edge between two nodes can be a joint embedding representation of a distance between two atoms represented by the two nodes and a type of the edge. The type of the edge can be one of four types: a target-ligand connecting edge, a target-target connecting edge, a ligand-target connecting edge, or a ligand-ligand connecting edge. Optionally, a fully connected graph can be constructed on the atoms in the ligand sample, which is represented by [symbol] _{L}. In the graph [symbol] _{L}, nodes represent atoms in the ligand sample, and node features are the aforementioned atom features [symbol] . The feature of an edge between two nodes includes the chemical bond type and the indication of whether the atoms come from the same prior distribution. The chemical bond type can be one of the following five types: single bond, double bond, triple bond, aromatic bond, or no chemical bond.

Optionally, a total number of the atom types can be represented as Kₐ, and a total number of the chemical bonds can be represented as K_{b}. It can be understood that the various types listed above are only illustrative. In an actual scenario, the total number of the types can be more or less. For example, in some examples, a set of the atom types can include seven types of atoms (namely, Kₐ=7). The present disclosure does not limit this.

The ligand generation model in the embodiments of the present disclosure is constructed based on a diffusion model, and the diffusion model involves a forward noise adding stage and a reverse denoising node. In a model training stage of the present disclosure, i.e., the process of generating the trained ligand generation model through training based on the decomposition prior distribution, the atom neighbor graph, and the fully connected graph, a noise adding stage and a denoising stage may be included. The noise adding stage and the denoising stage both include multiple steps. It is assumed that for step t, in the noise adding stage the step t is determined based on step t−1 and in the denoising stage the step t−1 is determined based on the step t.

In some embodiments, noised atom positions, noised atom types, and noised chemical bonds are obtained through the noise adding process based on the atom positions, the atom types, and the chemical bonds in the ligand sample.

Assuming that the atom positions, the atom types, and the chemical bonds at step t in the noise adding stage are represented by xₜ, vₜ and bₜ, respectively. Optionally, a corresponding Gaussian noise can be applied to the atom positions at step t−1, and corresponding white noises can be applied to the atom types and the chemical bonds at step t−1, respectively. For example, the method for applying the noise to each atom in the ligand sample can be expressed as the following formulas (1) to (3):

$$q ( {x}_{t} \vert {x}_{t - 1} , 𝒫 ) = {\sum }_{k = 1}^{K} {η}_{k} 𝒩 ( {\tilde{x}}_{t , k} ; {\tilde{x}}_{t - 1 , k} , {β}_{t} {\sum }_{k} ) \qquad ( 1 )$$

$$q ( {v}_{t} \vert {v}_{t - 1} , 𝒫 ) = C ( {v}_{t} \vert ( 1 - {β}_{t} ) {v}_{t - 1} + {β}_{t} / {K}_{a} ) \qquad ( 2 )$$

$$q ( {b}_{t} \vert {b}_{t - 1} , 𝒫 ) = C ( {b}_{t} \vert ( 1 - {β}_{t} ) {b}_{t - 1} + {β}_{t} / {K}_{b} ) \qquad ( 3 )$$

In formulas (1) to (3), q represents a noise adding function; [symbol] represents the atom positions, the atom types, and the chemical bonds of the target sample; [symbol] represents a Gaussian distribution; \tilde{x}_{t,k} represents the decentralized atom positions of the ligand, namely, \tilde{x}_{t,k}=xₜ−μₖ; ηₖ represents a prior distribution to which the atom belong, namely, {ηₖ∈{0,1}^{K}|Σₖ₌₁^{K}ηₖ=1}; C represents a discrete class distribution; and βₜ represents an amplitude of the noise applied in the noise adding process. Optionally, βₜ represents a preset known constant. Optionally, although the noise adding amplitudes in formulas (1) to (3) are all βₜ, different noise adding amplitudes can be used in practical applications. For example, βₜ in formula (1) may be not equal to βₜ in formula (2) or (3).

Assuming that the total number of steps is T, that is, a maximum value of t is T, for example, T=1000 or another value, the noised atom positions, the noised atom types, and the noised chemical bonds obtained in the noise adding stage can be expressed as x_{T}, v_{T}, and b_{T}, respectively.

In some embodiments, the atom neighbor graph and the fully connected graph are input to a group-equivariant graph neural network, and the noised atom positions, the noised atom types, and the noised chemical bonds are updated to obtain denoised atom positions, denoised atom types, and denoised chemical bonds.

Exemplarily, the group-equivariant graph neural network may include a plurality of (for example, six) modules stacked in sequence; each of the plurality of modules includes a first neural network, a second neural network, and a third neural network; the first neural network is configured to update a feature corresponding to the atom types; the second neural network is configured to update a feature corresponding to the chemical bonds; and the third neural network is configured to update a feature corresponding to the atom positions.

The group-equivariant graph neural network may be an SE(3) equivariant graph neural network. The first neural network, the second neural network, and the third neural network may be exemplarily expressed as φᵥ, φ_{b}, and φₓ in sequence. Exemplarily, the neighbor graph [symbol] _{K} and the fully connected graph [symbol] _{L} can be input to the SE(3) equivariant graph neural network to alternately update the feature corresponding to the atom types (referred to as an atom feature for example), the feature corresponding to the chemical bonds (referred to as a chemical bond feature for example), and the feature corresponding to the atom positions (i.e., the atom positions).

Exemplarily, the neighbor graph [symbol] _{K} and the fully connected graph [symbol] _{L} include the features of the nodes and the features of the edges, where the feature of the node can be represented as a scalar feature and a vector feature of the node (i.e., the atom). Specifically, an update process within a module can include: (1) integrating the various features (including the scalar features and vector features of the atoms and the features of the edges) through the first neural network φᵥ, so as to update the atom features; (2) integrating the various features (including the updated atom features (the scalar features and the vector features) and the features of the edges) through the second neural network φ_{b}, so as to update the chemical bond features; and (3) integrating the various features (including the updated atom features (the scalar features and the vector features) and the features of the edges) through the third neural network φₓ, so as to update the atom positions of the ligand. For example, in (1), the scalar features and vector features corresponding to the atoms in the target and the ligand can be extracted through the neural networks, and in (2), the chemical bond features between the atoms can be updated according to the feature of each atom in the ligand.

Optionally, the first neural network φᵥ, the second neural network φ_{b}, and the third neural network φₓ may each include an attention mechanism (such as a graph attention mechanism) for feature integration as described above. Optionally, the first neural network φᵥ, the second neural network φ_{b}, and the third neural network φₓ can also be referred to as three layers of the module, for example, which are respectively referred to as an atom feature update layer, a chemical bond feature update layer, and a position update layer. For example, a dimension of a feature vector in a layer can be set to be 128 or another, and a number of attention heads in a layer can be set to be 16 or another value.

In this way, through a plurality of (for example, six) modules, the updated atom positions, the updated atom types, and the updated chemical bonds can be obtained. Exemplarily, the updated atom positions processed by the plurality of modules are the denoised atom positions, denoted as \hat{x}. Exemplarily, the updated atom types processed by the plurality of modules can be then processed through several (such as two) multilayer perceptrons to obtain the denoised atom types, denoted as \hat{v}. Exemplarily, the updated chemical bonds processed by the plurality of modules can be then processed through several (such as two) multilayer perceptrons to obtain the denoised chemical bonds, denoted as \hat{b}.

It can be understood that the denoising stage involves multiple steps (such as T steps), where each step can include the update process using the SE(3) equivariant graph neural network comprising the plurality of modules, for example, representing an output of the step t and an input of the step t−1. Correspondingly, after the T steps, the denoised atom positions \hat{x}₀, the denoised atom types \hat{v}₀, and the denoised chemical bonds \hat{b}₀ can be obtained.

In some embodiments, a loss function can be constructed in the training process, and whether the training process is completed can be determined based on the loss function. Exemplarily, the loss function may include a first loss function, a second loss function, and a third loss function. Optionally, the first loss function, the second loss function, and the third loss function can be respectively expressed as Lₓ, Lᵥ, and L_{b}. The first loss function can be constructed based on the atom positions in the ligand sample and the denoised atom positions; the second loss function can be constructed based on the atom types in the ligand sample and the denoised atom types; and the third loss function can be constructed based on the chemical bonds in the ligand sample and the denoised chemical bonds. For example, the first loss function, the second loss function, and the third loss function can be obtained by the following formulas (4) to (6):

$${L}_{x} = {\sum }_{k = 1}^{K} {η}_{k} {‖ {\hat{x}}_{0} - {x}_{0} ‖}_{{\sum }_{k}^{- 1}} \qquad ( 4 )$$

$${L}_{v} = {\sum }_{k = 1}^{{K}_{a}} {c ( {v}_{t} , {v}_{0} )}_{k} log \frac{{c ( {v}_{t} , {v}_{0} )}_{k}}{{c ( {v}_{t} , {\hat{v}}_{0} )}_{k}} \qquad ( 5 )$$

$${L}_{b} = {\sum }_{k = 1}^{{K}_{b}} {c ( {b}_{t} , {b}_{0} )}_{k} log \frac{{c ( {b}_{t} , {b}_{0} )}_{k}}{{c ( {b}_{t} , {\hat{b}}_{0} )}_{k}} \qquad ( 6 )$$

In formula (5), c(vₜ, v₀) represents a posterior probability of the atom types, and in formula (6), c(bₜ, b₀) represents a posterior probability of the chemical bonds. Optionally, the loss function used for training can be determined by formula (7) below:

$$L = {L}_{x} + {γ}_{a} {L}_{v} + {γ}_{b} {L}_{b} \qquad ( 7 )$$

In formula (7), γₐ and γ_{b} represent weights. Exemplarily, in the training process, the weights can be updated using a gradient descent method until the convergence is achieved. For example, the gradient descent method may include a gradient descent algorithm of the Adam's adaptive learning rate.

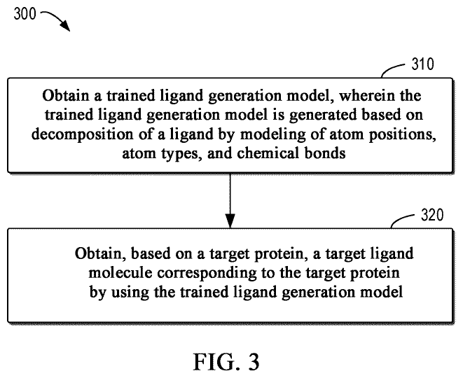

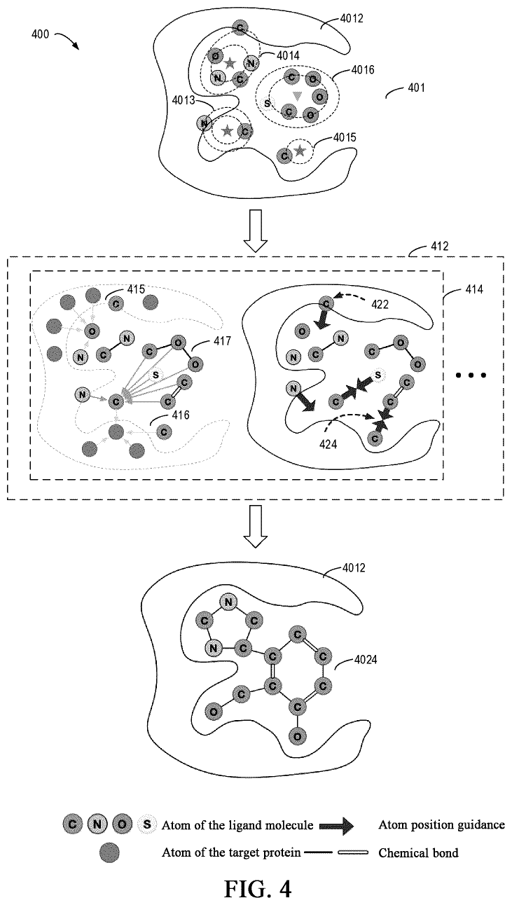

The exemplary training process of the ligand generation model has been described with reference to FIG. 2 above. The trained ligand generation model can be configured to generate a target ligand molecule corresponding to a target protein. In this training process, a prior distribution based on a decomposition characteristic of a ligand molecule is introduced and the chemical bonds are explicitly modeled. Specifically, in the embodiments of the present disclosure, generative modeling is performed on three kinds of information: the atom positions, the atom types, and the chemical bonds based on the diffusion model. By decomposing the atoms into the plurality of clusters and considering the chemical bond types between the atoms, the affinity between the ligand and the target can be improved more explicitly, such that the problem of insufficient rationality can be alleviated. A schematic use process of the ligand generation model will be described in conjunction with FIG. 3 and FIG. 4 below.

FIG. 3 shows a flowchart of an exemplary use process 300 according to some embodiments of the present disclosure. At block 310, a trained ligand generation model is obtained, wherein the trained ligand generation model is generated based on decomposition of a ligand by modeling of atom positions, atom types, and chemical bonds. At block 320, a target ligand molecule corresponding to a target protein is obtained based on the target protein by using the trained ligand generation model.

It can be understood that the trained ligand generation model can be obtained through the training in the training process shown in FIG. 2. In the embodiments of the present disclosure, the target protein may be input by a user or obtained from a storage device.

Exemplarily, the trained ligand generation model can be expressed as p(M₀| [symbol] ), where [symbol] represents the initial data that is input to the model, and M₀=(x₀, v₀, b₀) represents the output data that is output by the model.

In some embodiments of the present disclosure, the initial data may be determined based on the target protein. For example, the initial data include initial atom positions, initial atom types, and initial chemical bonds. Later, the initial data is input to the trained ligand generation model to obtain the output data. For example, the output data includes output atom positions, output atom types, and output chemical bonds. Exemplarily, the output data may be the target ligand molecule corresponding to the target protein.

Exemplarily, a decomposition prior distribution of a scaffold and arms in the initial ligand molecule can be determined based on the target protein. Afterwards, the initial data can be determined by sampling based on the decomposition prior distribution. Optionally, the decomposition prior distribution includes mean matrixes and covariance matrixes of a plurality of clusters. For example, the decomposition prior distribution includes a plurality of prior distributions corresponding to the plurality of clusters, and each of the prior distributions includes the mean matrix and covariance matrix of the respective cluster.

In some embodiments, whether the target protein has a known ligand molecule can be determined. If there is any, the known ligand molecule may be used as the initial ligand molecule. Exemplarily, the known ligand molecule that binds to the target protein can be obtained; a plurality of atoms of the known ligand molecule are decomposed into a plurality of clusters based on binding situations between the plurality of atoms and a binding pocket of the target protein, wherein one cluster among the plurality of clusters serves as a scaffold cluster, and the remaining clusters among the plurality of clusters serve as arm clusters; the mean matrix and covariance matrix of each of the plurality of clusters are determined based on coordinates of the atoms in each of the plurality of clusters; and the decomposition prior distribution is determined based on the mean matrixes and covariance matrixes of the plurality of clusters. It can be understood that the process of determining the decomposition prior distribution based on the target protein and the known ligand molecule is similar to the process of determining the decomposition prior distribution based on the target sample and the ligand sample in the above training process, and will not be elaborated here for simplicity.

If there isn't any known ligand molecule, a pseudo ligand molecule can be determined first, and then the pseudo ligand molecule may be used as the initial ligand molecule. Exemplarily, the pseudo ligand molecule that binds to the target protein can be determined by prediction. For example, the pseudo ligand molecule can be obtained through a prediction algorithm, such as using AlphaSpace2. Further, the decomposition prior distribution can be determined based on the pseudo ligand molecule. Exemplarily, a plurality of pseudo ligand atoms in the pseudo ligand molecule can be determined based on a binding pocket of the target protein; for example, the plurality of pseudo ligand atoms are Beta atoms; the plurality of pseudo ligand atoms are decomposed into a plurality of clusters based on a distance between every two of the plurality of pseudo ligand atoms, wherein one cluster among the plurality of clusters serves as a scaffold cluster, and the remaining clusters among the plurality of clusters serve as arm clusters; the mean matrix and covariance matrix of each of the plurality of clusters are determined based on coordinates of the atoms in each of the plurality of clusters; and the decomposition prior distribution is determined based on the mean matrixes and covariance matrixes of the plurality of clusters. For example, the Beta atoms can be retrieved from the vicinity of the binding pocket of the target protein, and a Beta score can be calculated to indicate an ability of the corresponding Beta atom being paired to the binding pocket. The Beta atoms can be classified to the plurality of clusters through clustering based on the distances between the Beta atoms. For example, two Beta atoms with a distance less than a threshold (10 Å) belong to the same cluster. Further, a mean value of the Beta scores of all the Beta atoms in the same cluster can be a Beta score of the cluster. Then the plurality of clusters can be classified to a scaffold cluster and arm clusters through filtering. The mean matrix and covariance matrix of each cluster are determined to obtain the decomposition prior distribution.

In some embodiments, the initial data can be determined by sampling. Exemplarily, the decomposition prior distribution can be sampled first to determine the initial atom positions; a set of the plurality of atom types is uniformly sampled to determine the initial atom types; and a set of the plurality of chemical bonds is uniformly sampled to determine the initial chemical bonds. For example, the initial atom positions, the initial atom types, and the initial chemical bonds are expressed as xₜ, vₜ and bₜ, and satisfy x_{T}˜ [symbol] (μₖ,Σₖ), v_{T}˜Uniform(Kₐ), and b_{T}˜Uniform(K_{b}) respectively, where “Uniform” represents a function that randomly performs uniform sampling.

In some embodiments, the process of obtaining the output data may include: inputting the initial data to the trained ligand generation model, and obtaining the output data through a multi-step denoising process. Exemplarily, at each step of the multi-step denoising process: the atom positions, atom types, and chemical bonds obtained in the previous step are input to the group-equivariant graph neural network, and intermediate atom positions, intermediate atom types, and intermediate chemical bonds are obtained through denoising; and a position gradient guidance is applied to the intermediate atom positions to obtain atom positions, atom types, and chemical bonds for a next step based on the intermediate atom positions, the intermediate atom types, and the intermediate chemical bonds.

If the multi-step denoising process involves T steps, an input of step t−1 can be determined based on an output of step t. Assuming that the output of the step t includes xₜ, vₜ, bₜ, it can be output to the SE(3) equivariant graph neural network for denoising the atom positions, the atom types, and the chemical bonds to obtain \hat{x}, \hat{v} and \hat{b}. As shown in the training process combined with FIG. 2, the SE(3) equivariant graph neural network includes a plurality of modules, each of which includes three layers, i.e., the first neural network φᵥ, the second neural network φ_{b}, and the third neural network φₓ. The process of obtaining \hat{x}, \hat{v} and \hat{b} is similar to the corresponding process in the previous training process, and will not be elaborated here for simplicity.

Exemplarily, a position guidance can be further applied based on obtaining \hat{x}, \hat{v} and \hat{b} through the plurality of modules of the SE(3) equivariant graph neural network. Exemplarily, the position gradient guidance is configured to apply a position constraint on the basis of the intermediate atom positions, so that a distance between a scaffold atom and an arm atom that bonds to the scaffold atom is between a minimum chemical bond length and a maximum chemical bond length, and the scaffold atom and the arm atom do not collide with atoms of the target protein.

The position guidance can also be referred to as a rationality guidance. The quality and accuracy of the generated target ligand molecule can be improved by introducing the rationality guidance. In some examples, the gradient guidance as shown in formula (8) can be applied to the atom positions based on the chemical bond length:

$$- {∇}_{{x}_{t}} {\sum }_{n = 1}^{\vert 𝒜 \vert} [ {ξ}_{2} max ( 0 , {d}_{t}^{( n )} - {ρ}_{max} ) + {ξ}_{1} max ( 0 , {ρ}_{min} - {d}_{t}^{( n )} ) ] \qquad ( 8 )$$

dₜ⁽ⁿ⁾ in formula (8) is defined in the following formula (9):

$${d}_{t}^{( n )} = {min}_{i ∈ {𝒜}_{n} , j ∈ 𝒮} ‖ {x}_{t}^{( i )} - {x}_{t}^{( j )} ‖ \qquad ( 9 )$$

In formulas (8) and (9), ∇ₓ_{ₜ} represents a gradient for xₜ; dₜ⁽ⁿ⁾ represents a distance between the atoms at step t; [symbol] represents a set of the arm atoms; [symbol] ₙ represents a set of the ligand atoms included in an n-th arm cluster; [symbol] represents a set of the scaffold atoms; ρₘᵢₙ and ρₘₐₓ represent a minimum value and a maximum value of the reasonable chemical bond length, respectively; and ξ₁ and ξ₂ are hyperparameters greater than 0.

In this way, by applying a constraint as shown in formula (8) to the atom positions, the distance between the arm atom and the scaffold atom in the ligand molecule can be the reasonable chemical bond length, thereby encouraging the arm atoms and the scaffold atoms to be connected to form a complete molecule.

In some examples, the gradient guidance as shown in the following formula (10) can be applied to the atom positions of the ligand based on the atoms of the target protein:

$$- {∇}_{{x}_{t}} {ξ}_{3} {\sum }_{i}^{{N}_{M}} max ( 0 , γ - S ( {x}_{t}^{( i )} ) ) \qquad ( 10 )$$

S in formula (10) is defined in the following formula (11):

$$S ( x ) = - σ ln {\sum }_{j = 1}^{{N}_{P}} exp ( - {‖ x - {x}_{P}^{( j )} ‖}^{2} / σ ) \qquad ( 11 )$$

In formulas (10) and (11), σ, γ, and ξ₃ are hyperparameters; x_{P}^{(j)} represents the position of a j-th atom of the target protein; N_{P} represents the total number of the atoms of the target protein; and N_{M} represents the total number of the atoms of the ligand molecule.

In this way, by applying the constraint as shown in formula (10) to the atom positions, it is possible to avoid spatial collisions between the atoms in the generated ligand molecule and the target protein.

In some examples, xₜ₋₁, vₜ₋₁, bₜ₋₁ to be used at step t−1 can be obtained by applying the constraint to the atom positions based on \hat{x}, \hat{v}, and \hat{b} at step/among the T steps.

Correspondingly, it can be understood that the similar operations can be performed for each step from step T to step 0, and the output data M₀=(x₀, v₀, b₀) can be obtained. Afterwards, molecular construction can be performed based on the output atom positions x₀, the output atom types v₀, and the output chemical bonds b₀ to obtain the target ligand molecule.

In some examples, the model use process 300 can also be referred to as a model inference process, a DecompDiff sampling process, or the like, and the present disclosure does not limit this.

FIG. 4 shows a schematic diagram of generating a target ligand molecule 400 according to some embodiments of the present disclosure. As shown in FIG. 4, for a target protein 4012, an initial ligand molecule (such as a known ligand molecule or a pseudo ligand molecule, also referred to as an initial ligand molecule) can be determined, and a decomposition prior distribution 401 of the initial ligand molecule can be determined, for example, including a plurality of prior distributions corresponding to a plurality of clusters. In FIG. 4, a plurality of clusters are marked by ellipses, for example, including a plurality of arm clusters 4013 to 4015 and a scaffold cluster 4016. The pentagram located in a center of the ellipse represents a mean (prior center) of the arm cluster, and the inverted triangle in a center of the ellipse represents a mean (prior center) of the scaffold cluster.

Further, the corresponding output data can be obtained through a trained ligand generation model 412. This process involves multiple steps (such as T=1000), where an output of step t is an input of step t−1. As an example, step T 414 is shown in FIG. 4.

At step T 414, a neighbor graph and a fully connected diagram can be determined based on the target protein 4012 and the decomposition prior distribution 401 of the initial ligand molecule. For example, nodes in the neighbor graph 415-416 include atoms in the target protein 4012 and atoms in the initial ligand molecule. For example, nodes in the fully connected diagram 417 are the atoms in the initial ligand molecule. Furthermore, at step T 414, a position guidance is also applied to atom positions. For example, at 422, there may be a collision between the ligand atoms and the atoms in the target protein 4012, thus a position constraint can be applied to keep the ligand atoms away from the atoms in the target protein 4012. For example, at 424, a distance between two ligand atoms may be greater than the maximum value of the chemical bond length, thus the position constraint can be applied to reduce the distance between the two atoms.

After the T steps, the output data can be obtained. For example, ligand molecule 4024 can be determined. Furthermore, it can be seen that compared with the initial ligand molecule at 401, the ligand molecule 4024 obtained through the trained ligand generation model according to the embodiments of the present disclosure has the higher affinity with the target protein 4012.

TABLE 1

| | Score value |
|---|---|
| Baseline model | −7.8 |
| Model of the present disclosure | −8.39 |

Table 1 shows example results of effects of the scheme according to the embodiments of the present disclosure. The baseline model in Table 1 represents a target diffusion model (TargetDiff), which models atom positions and atom types based on the diffusion model. The score values in Table 1 are average scores of Vina Docking Scores on the CrossDocked 2020 dataset. As the lower the score value, the better the model, it can be seen that this scheme (with the score value of −8.39) is superior to the baseline model (with the score value of −7.8). Therefore, the scheme according to the embodiments of the present disclosure can obtain the target ligand molecules with the higher affinity.

In addition, the scheme according to the embodiments of the present disclosure has a higher proportion of meeting the requirements for drug-likeness and synthesizability of finished drugs. For example, on the CrossDocked 2020 dataset, an average quantitative estimate of drug-likeness (QED) of 0.45 and an average synthetic accessibility (SA) of 0.61 can be achieved. An overall considered success rate indicator for the Vina Docking Score, the QED, and the SA can reach 24.5%. Therefore, the scheme according to the embodiments of the present disclosure has a certain degree of rationality.

In this way, the embodiments of the present disclosure provide a scheme for ligand generation, which can obtain the target ligand molecule corresponding to the target protein based on the trained ligand generation model. Exemplarily, in the embodiments of the present disclosure, the decomposition prior distribution is determined as the input and thus, for example, the Vina Docking Score can be increased to −9.08. Exemplarily, in the embodiments of the present disclosure, modeling the chemical bonds can significantly improve the QED and the SA, for example, the success rate can be increased to 15.38%. Exemplarily, in the embodiments of the present disclosure, the guidance/constraint is applied to the atom positions, so that the Vina Docking Score can be increased to −6.11, and the completion ratio can be increased 0.94, where the completion ratio represents a probability of all the atoms in the generated ligand to be connected to form a legitimate molecule.

It should be understood that in the embodiments of the present disclosure, the terms “first”, “second”, “third”, and the like are only used to indicate that multiple objects may be different, but at the same time, it is not ruled out that two objects are the same. They should not be interpreted as any limitation on the embodiments of the present disclosure.

It should also be understood that the methods, situations, categories, and division of the embodiments in the embodiments of the present disclosure are only for the convenience of description and should not constitute special limitations. Various methods, categories, situations, and features in the embodiments can be combined with each other in a logical manner.

It should also be understood that the above content is only intended to help those skilled in the art better understand the embodiments of the present disclosure, and is not intended to limit the scope of the embodiments of the present disclosure. Those skilled in the art can make various modifications, changes, or combinations according to the above content. These modified, changed, or combined solutions also fall within the scope of the embodiments of the present disclosure.

It should also be understood that the description of the above content emphasizes differences between all the embodiments, and identical or similar parts can be used for reference. For simplicity, they will not be elaborated here.

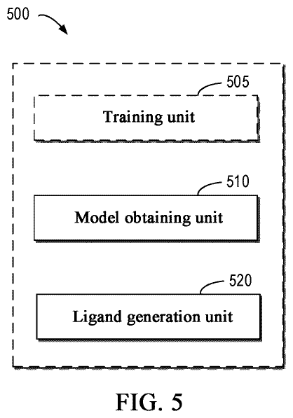

FIG. 5 shows a block diagram of an exemplary apparatus 500 according to some embodiments of the present disclosure. The apparatus 500 may be implemented by software, hardware, or a combination of software and hardware. As shown in FIG. 5, the apparatus 500 includes a model obtaining unit 510 and a ligand generation unit 520.

The model obtaining unit 510 is configured to obtain a trained ligand generation model, wherein the trained ligand generation model is generated based on decomposition of a ligand by modeling of atom positions, atom types, and chemical bonds. The ligand generation unit 520 is configured to obtain, based on a target protein, a target ligand molecule corresponding to the target protein by using the trained ligand generation model.

In some embodiments, the ligand generation unit 520 may include a decomposition prior distribution determining unit, an initial data determining unit, an output data determining unit, and a target ligand generation unit. The decomposition prior distribution determining unit is configured to determine a decomposition prior distribution of a scaffold and arms in an initial ligand molecule based on the target protein, wherein the decomposition prior distribution includes mean matrixes and covariance matrixes of a plurality of clusters. The initial data determining unit is configured to determine initial data based on the decomposition prior distribution by sampling, wherein the initial data includes initial atom positions, initial atom types, and initial chemical bonds. The output data determining unit is configured to input the initial data to the trained ligand generation model to obtain output data, wherein the output data includes output atom positions, output atom types, and output chemical bonds. The target ligand generation unit is configured to generate the target ligand molecule based on the output data.

The initial ligand molecule may include a known ligand molecule that binds to the target protein, and the decomposition prior distribution determining unit can be configured to: obtain the known ligand molecule that binds to the target protein; decompose a plurality of atoms of the known ligand molecule into a plurality of clusters based on binding situations between the plurality of atoms and a binding pocket of the target protein, wherein one cluster among the plurality of clusters serves as the scaffold, and the remaining clusters among the plurality of clusters serve as the arms; determine the mean matrix and covariance matrix of each of the plurality of clusters based on coordinates of the atoms in each of the plurality of clusters; and determine the decomposition prior distribution based on the mean matrixes and covariance matrixes of the plurality of clusters.

The initial ligand molecule may include a pseudo ligand molecule that binds to the target protein, and the decomposition prior distribution determining unit can be configured to: determine the pseudo ligand molecule that binds to the target protein by prediction; determine a plurality of pseudo ligand atoms in the pseudo ligand molecule based on a binding pocket of the target protein; decompose the plurality of pseudo ligand atoms into a plurality of clusters based on a distance between every two of the plurality of pseudo ligand atoms, wherein one cluster among the plurality of clusters serves as the scaffold, and the remaining clusters among the plurality of clusters serve as the arms; determine the mean matrix and covariance matrix of each of the plurality of clusters based on coordinates of the atoms in each of the plurality of clusters; and determine the decomposition prior distribution based on the mean matrixes and covariance matrixes of the plurality of clusters.

Exemplarily, the initial data determining unit can be configured to: sample the decomposition prior distribution to determine the initial atom positions; uniformly sample a set of a plurality of atom types to determine the initial atom types; and uniformly sample a set of a plurality of chemical bonds to determine the initial chemical bonds.

Exemplarily, the output data determining unit can be configured to: input the initial data to the trained ligand generation model, and obtain the output data through a multi-step denoising process.

The trained ligand generation model includes a group-equivariant graph neural network, and the output data determining unit can be configured to: at each step of the multi-step denoising process, input the atom positions, atom types, and chemical bonds obtained in the previous step to the group-equivariant graph neural network, and obtain intermediate atom positions, intermediate atom types, and intermediate chemical bonds through denoising; and apply a position gradient guidance to the intermediate atom positions to obtain atom positions, atom types, and chemical bonds for a next step based on the intermediate atom positions, the intermediate atom types, and the intermediate chemical bonds.

Optionally, the position gradient guidance is configured to apply a position constraint on the basis of the intermediate atom positions, so that a distance between a scaffold atom and an arm atom that bonds to the scaffold atom is between a minimum chemical bond length and a maximum chemical bond length, and the scaffold atom and the arm atom do not collide with atoms of the target protein.

As shown in FIG. 5, the apparatus 500 can further include a training unit 505, configured to: obtain a training dataset that includes a plurality of training data items, wherein each of the plurality of training data items includes a target sample and a corresponding ligand sample; and generate the trained ligand generation model through training based on the training dataset.

In some embodiments, the training unit 505 can be specifically configured to: determine a decomposition prior distribution based on the target sample and the ligand sample; construct an atom neighbor graph, wherein the atom neighbor graph represents relationships between a plurality of atoms that are near to each other; construct a fully connected graph, wherein the fully connected graph represents relationships between different atoms of the ligand sample; and generate the trained ligand generation model by training based on the decomposition prior distribution, the atom neighbor graph, and the fully connected graph.

The trained ligand generation model includes a group-equivariant graph neural network, and the training unit 505 can be specifically configured to: obtain noised atom positions, noised atom types, and noised chemical bonds through a noise adding process based on atom positions, atom types, and chemical bonds in the ligand sample; and input the atom neighbor graph and the fully connected graph to the group-equivariant graph neural network, and update the noised atom positions, the noised atom types, and the noised chemical bonds to obtain denoised atom positions, denoised atom types, and denoised chemical bonds.

Exemplarily, the group-equivariant graph neural network includes a plurality of modules stacked in sequence; each of the plurality of modules includes a first neural network, a second neural network, and a third neural network; the first neural network is configured to update a feature corresponding to the atom types; the second neural network is configured to update a feature corresponding to the chemical bonds; and the third neural network is configured to update a feature corresponding to the atom positions.

Optionally, a feature of a node corresponding to the atom of the target sample in the atom neighbor graph includes at least one of the following of the atom: the atom type, the atom position, amino acid type, the indication of whether the atom is located on a main chain, and the indication of one or more arms with the atom being located in a predetermined range therein; a feature of a node corresponding to the atom of the ligand sample in the atom neighbor graph include at least one of the following of the atom: the atom type, the atom position, and the indication of an arm or a scaffold that the atom belongs to; a feature of an edge of the atom neighbor graph includes a distance between two atoms connected by the edge and a type of the edge; and the type of the edge includes any of the following: a target and ligand connecting edge, a target and target connecting edge, a ligand and target connecting edge, or a ligand and ligand connecting edge.

Optionally, a feature of a node of the fully connected graph includes at least one of the following of an atom: the atom type, the atom position, an indication of whether the atom belongs to a scaffold or an arm; and a feature of an edge of the fully connected graph includes the chemical bond type and an indication of whether two atoms connected by the edge belong to the same decomposition prior distribution.

Exemplarily, the training unit 505 is further configured to determine, based on a loss function, whether a training process is completed, wherein the loss function includes a first loss function constructed based on the atom positions in the ligand sample and the denoised atom positions, a second loss function constructed based on the atom types in the ligand sample and the denoised atom types, and a third loss function constructed based on the chemical bonds in the ligand sample and the denoised chemical bonds.

The apparatus 500 in FIG. 5 can be configured to implement the processes described in combination with FIG. 2 to FIG. 4. For simplicity, it will not be elaborated here.

The division of the modules or units in the embodiments of the present disclosure is illustrative and is only a logical functional division. In actual implementation, there may also be another division method. In addition, in the embodiments of the present disclosure, all the functional units can be integrated into one unit or can exist physically alone, or two or more units can be integrated into one unit. The integrated units mentioned above can be implemented in both a hardware form and a software functional unit form.

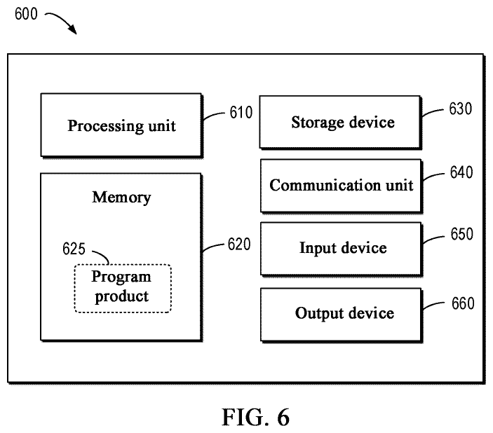

FIG. 6 shows a block diagram of an exemplary device 600 configured to implement an embodiment of the present disclosure. It should be understood that the device 600 shown in FIG. 6 is only exemplary and should not impose any limitation on the functionality and scope of the implementations described herein. For example, the device 600 can be used to perform the processes described in FIG. 2 to FIG. 4 above. For example, the device 600 can be implemented as a classical computer and/or a quantum computer.

As shown in FIG. 6, the device 600 is in the form of a general-purpose computing device. Components of the computing device 600 may include, but are not limited to, one or more processors or processing units 610, a memory 620, a storage device 630, one or more communication units 640, one or more input devices 650, and one or more output devices 660. The processing unit 610 can be an actual or virtual processor and can execute various processing based on programs stored in the memory 620. In a multiprocessor system, the plurality of processing units execute computer-executable instructions in parallel to improve the parallel processing capability of the computing device 600.

The computing device 600 usually includes a plurality of computer storage media. These media can be any accessible media that the computing device 600 can access, including but not limited to volatile and non-volatile media, and removable and non-removable media. The memory 620 can be a volatile memory (such as a register, a cache, a Random Access Memory (RAM)), a non-volatile memory (such as a Read Only Memory (ROM), an Electrically Erasable Programmable Read Only Memory (EEPROM), and a flash memory), or a combination of them. The storage device 630 may be a removable or non-removable medium, and may include machine-readable media, such as a flash drive, a hard disk drive, or any other media, can be configured to store information and/or data (such as training data for training), and can be accessed in the computing device 600.

The computing device 600 may further include other removable/non-removable, volatile/non-volatile storage media. Although not shown in FIG. 6, a hard disk drive for reading from or writing in removable, non-volatile magnetic disks (such as a “floppy disk”) and an optical disk drive for reading from or writing in from removable, non-volatile optical disk can be provided. In these cases, each drive can be connected to the bus by one or more data medium interfaces (not shown). The memory 620 may include a computer program product 625, which has one or more program modules, and these program modules are configured to perform various methods or actions of the various implementations of the present disclosure.

The communication unit 640 achieves communication with other computing devices through communication media. Additionally, the functions of the components of the computing device 600 can be implemented in a single computing cluster or multiple computing machines, and these computing machines can communicate through communication connections. Therefore, the computing device 600 can be operated in a networked environment using a logical connection with one or more other servers, network personal computers (PCs), or another network node.

The input device 650 can be one or more input devices, such as a mouse, a keyboard, and a trackball. The output device 660 can be one or more output devices, such as a display, a speaker, and a printer. The computing device 600 can also communicate with one or more external devices (not shown) as needed through the communication unit 640. The external device, such as a storage device and a display device, communicates with one or more devices that enable a user to interact with the computing device 600, or with any device (such as a network card and a modem) that enables the computing device 600 to communicate with one or more other computing devices. This communication can be executed through an input/output (I/O) interface (not shown).

According to an exemplary implementation of the present disclosure, a computer-readable storage medium is provided, which stores computer-executable instructions. The computer-executable instructions are executed by a processor to implement the method described above. According to an exemplary implementation of the present disclosure, a computer program product is further provided. The computer program product is tangibly stored on a non-transitory computer-readable medium and includes computer-executable instructions. The computer-executable instructions are executed by a processor to implement the method described above. According to an exemplary implementation of the present disclosure, a computer program product is provided, which stores a computer program. The program, when executed by a processor, implements the method described above.

The flowchart and/or block diagram of the method, apparatus, device, and computer program product implemented according to the present disclosure describe all the aspects of the present disclosure. It should be understood that each block of the flowchart and/or block diagram and combinations of all the blocks in the flowchart and/or block diagram can be implemented by the computer-readable program instructions.

These computer-readable program instructions may be provided to a general-purpose computer, a dedicated computer, or a processing unit of another programmable data processing apparatus to generate a machine, so that these instructions, when executed by the computer or the processing unit of the another programmable data processing apparatus, generate an apparatus for implementing a specific function/action in one or more flows in the flowcharts and/or in one or more blocks in the flowchart and/or block diagram. These computer-readable program instructions can also be stored in the computer-readable storage medium, and these instructions enable the computer, the programmable data processing apparatus, and/or other devices to work in a specific way. Therefore, the computer-readable medium storing the instructions includes a manufacture, which includes instructions for implementing various aspects of the functions/actions specified in one or more blocks in the flowchart and/or block diagram.

The computer-readable program instructions can be loaded onto the computer, the another programmable data processing apparatus, or the another device to perform a series of operational steps on the computer, the another programmable data processing apparatus, or the another device, so as to generate a computer-implemented process. The instructions executed on the computer, the another programmable data processing apparatus, or the another device implement the functions/actions specified in one or more blocks in the flowchart and/or block diagram.

The flowcharts and block diagrams in the accompanying drawings show possible system architectures, functions, and operations of the system, method, and computer program product of a plurality of implementations of the present disclosure. In this regard, each block in the flowchart or block diagram may represent a module, a program, or a part of an instruction. The module, the program, or the part of the instruction includes one or more executable instructions used for implementing specified logic functions. In some implementations used as substitutes, functions annotated in blocks may alternatively occur in a sequence different from that annotated in an accompanying drawing. For example, actually two blocks shown in succession may be performed basically in parallel, and sometimes the two blocks may be performed in a reverse sequence. This is determined by a related function. It is also noted that each block in the block diagram or the flowchart and a combination of the blocks in the block diagram and/or the flowchart may be implemented by using a dedicated hardware-based system configured to perform a specified function or operation, or may be implemented by using a combination of dedicated hardware and computer instructions.

The above has described the various implementations of the present disclosure. The above explanation is exemplary, not exhaustive, and is not limited to the various implementations disclosed herein. Many modifications and changes are obvious to those of ordinary skill in the art without deviating from the scope and spirit of the various implementations described herein. The selection of the terms used herein aims to best explain the principles and practical applications of the various implementations or improvements to technologies in the market, or to enable other persons of ordinary skill in the art to understand the various implementations disclosed herein.

I/We claim:

1. A method for ligand generation, comprising:
obtaining a trained ligand generation model, wherein the trained ligand generation model is generated based on decomposition of a ligand by modeling of atom positions, atom types, and chemical bonds; and
obtaining, based on a target protein, a target ligand molecule corresponding to the target protein by using the trained ligand generation model.

2. The method according to claim 1, wherein obtaining, based on the target protein, the target ligand molecule corresponding to the target protein by using the trained ligand generation model comprises:
determining, based on the target protein, a decomposition prior distribution of a scaffold and arms in an initial ligand molecule, wherein the decomposition prior distribution comprises mean matrixes and covariance matrixes of a plurality of clusters;
determining initial data based on the decomposition prior distribution by sampling, wherein the initial data comprises initial atom positions, initial atom types, and initial chemical bonds;
inputting the initial data to the trained ligand generation model to obtain output data, wherein the output data comprises output atom positions, output atom types, and output chemical bonds; and
generating the target ligand molecule based on the output data.

3. The method according to claim 2, wherein the initial ligand molecule comprises a known ligand molecule that binds to the target protein, and wherein determining, based on the target protein, the decomposition prior distribution of the scaffold and arms in the initial ligand molecule comprises:
obtaining the known ligand molecule that binds to the target protein;
decomposing, based on a binding situation between a plurality of atoms of the known ligand molecule and a binding pocket of the target protein, the plurality of atoms into a plurality of clusters, wherein one cluster among the plurality of clusters serves as the scaffold, and the remaining clusters among the plurality of clusters serve as the arms;
determining, based on coordinates of the atoms in each of the plurality of clusters, the mean matrix and covariance matrix of each of the plurality of clusters; and
determining the decomposition prior distribution based on the mean matrixes and covariance matrixes of the plurality of clusters.

4. The method according to claim 2, wherein the initial ligand molecule comprises a pseudo ligand molecule that binds to the target protein, and wherein determining, based on the target protein, the decomposition prior distribution of the scaffold and arms in the initial ligand molecule comprises:
determining the pseudo ligand molecule that binds to the target protein by a prediction;
determining, based on a binding pocket of the target protein, a plurality of pseudo ligand atoms in the pseudo ligand molecule;
decomposing, based on a distance between every two of the plurality of pseudo ligand atoms, the plurality of pseudo ligand atoms into a plurality of clusters, wherein one cluster among the plurality of clusters serves as the scaffold, and the remaining clusters among the plurality of clusters serve as the arms;
determining, based on coordinates of the atoms in each of the plurality of clusters, the mean matrix and covariance matrix of each of the plurality of clusters; and
determining the decomposition prior distribution based on the mean matrixes and covariance matrixes of the plurality of clusters.

5. The method according to claim 2, wherein determining the initial data based on the decomposition prior distribution by sampling comprises:
sampling the decomposition prior distribution to determine the initial atom positions;
uniformly sampling a set of a plurality of atom types to determine the initial atom types; and
uniformly sampling a set of a plurality of chemical bonds to determine the initial chemical bonds.

6. The method according to claim 2, wherein inputting the initial data to the trained ligand generation model to obtain output data comprises:
inputting the initial data to the trained ligand generation model, and obtaining the output data through a multi-step denoising process.

7. The method according to claim 6, wherein the trained ligand generation model comprises a group-equivariant graph neural network, and each step of the multi-step denoising process comprises:
inputting atom positions, atom types, and chemical bonds obtained in a previous step to the group-equivariant graph neural network, and obtaining intermediate atom positions, intermediate atom types, and intermediate chemical bonds through denoising; and
obtaining atom positions, atom types, and chemical bonds for a next step based on the intermediate atom positions, the intermediate atom types, and the intermediate chemical bonds by applying a position gradient guidance to the intermediate atom positions.

8. The method according to claim 7, wherein the position gradient guidance is configured to apply a position constraint on the basis of the intermediate atom positions, so that a distance between a scaffold atom and an arm atom that bonds to the scaffold atom is between a minimum chemical bond length and a maximum chemical bond length, and the scaffold atom and the arm atom do not collide with atoms of the target protein.

9. The method according to claim 1, further comprising:
obtaining a training dataset that comprises a plurality of training data items, wherein each of the plurality of training data items comprises a target sample and a corresponding ligand sample; and
generating the trained ligand generation model through training based on the training dataset.

10. The method according to claim 9, wherein generating the trained ligand generation model through training comprises:
determining a decomposition prior distribution based on the target sample and the ligand sample;
constructing an atom neighbor graph, wherein the atom neighbor graph represents relationships between a plurality of atoms that are near to each other;
constructing a fully connected graph, wherein the fully connected graph represents relationships between different atoms of the ligand sample; and
generating the trained ligand generation model by training based on the decomposition prior distribution, the atom neighbor graph, and the fully connected graph.

11. The method according to claim 10, wherein the trained ligand generation model comprises a group-equivariant graph neural network, and wherein generating the trained ligand generation model by training based on the decomposition prior distribution, the atom neighbor graph, and the fully connected graph comprises:
obtaining noised atom positions, noised atom types, and noised chemical bonds through a noise adding process based on atom positions, atom types, and chemical bonds in the ligand sample; and
inputting the atom neighbor graph and the fully connected graph to the group-equivariant graph neural network, and updating the noised atom positions, the noised atom types, and the noised chemical bonds to obtain denoised atom positions, denoised atom types, and denoised chemical bonds.

12. The method according to claim 11, wherein the group-equivariant graph neural network comprises a plurality of modules stacked in sequence; each of the plurality of modules comprises a first neural network, a second neural network, and a third neural network; and wherein the first neural network is configured to update a feature corresponding to the atom types, the second neural network is configured to update a feature corresponding to the chemical bonds, and the third neural network is configured to update a feature corresponding to the atom positions.

13. The method according to claim 10, wherein a feature of a node corresponding to an atom of the target sample in the atom neighbor graph comprises at least one of the following of the atom: an atom type, an atom position, an amino acid type, an indication of whether the atom is located on a main chain, and an indication of one or more arms with the atom being located in a predetermined range therein; wherein a feature of a node corresponding to an atom of the ligand sample in the atom neighbor graph comprises at least one of the following of the atom: an atom type, an atom position, and an indication of which arm or which scaffold that the atom belongs to; wherein a feature of an edge of the atom neighbor graph comprises a distance between two atoms connected by the edge and a type of the edge; and wherein the type of the edge comprises any of the following: a target and ligand connecting edge, a target and target connecting edge, a ligand and target connecting edge, or a ligand and ligand connecting edge.

14. The method according to claim 10, wherein a feature of a node of the fully connected graph comprises at least one of the following of an atom: an atom type, an atom position, an indication of whether the atom belongs to a scaffold, or an indication of whether the atom belongs to the arms; and wherein a feature of an edge of the fully connected graph comprises a chemical bond type and an indication of whether two atoms connected by the edge belong to the same decomposition prior distribution.

15. The method according to claim 11, further comprising:
determining, based on a loss function, whether a training process is completed, wherein the loss function comprises a first loss function constructed based on the atom positions in the ligand sample and the denoised atom positions, a second loss function constructed based on the atom types in the ligand sample and the denoised atom types, and a third loss function constructed based on the chemical bonds in the ligand sample and the denoised chemical bonds.

16. An electronic device, comprising:
at least one processing unit; and
at least one memory, wherein the at least one memory is coupled to the at least one processing unit and stores instructions executed by the at least one processing unit, and the instructions, when executed by the at least one processing unit, cause the electronic device to perform actions comprising:
obtaining a trained ligand generation model, wherein the trained ligand generation model is generated based on decomposition of a ligand by modeling of atom positions, atom types, and chemical bonds; and
obtaining, based on a target protein, a target ligand molecule corresponding to the target protein by using the trained ligand generation model.

17. The electronic device according to claim 16, wherein obtaining, based on the target protein, the target ligand molecule corresponding to the target protein by using the trained ligand generation model comprises:
determining, based on the target protein, a decomposition prior distribution of a scaffold and arms in an initial ligand molecule, wherein the decomposition prior distribution comprises mean matrixes and covariance matrixes of a plurality of clusters;
determining initial data based on the decomposition prior distribution by sampling, wherein the initial data comprises initial atom positions, initial atom types, and initial chemical bonds;
inputting the initial data to the trained ligand generation model to obtain output data, wherein the output data comprises output atom positions, output atom types, and output chemical bonds; and
generating the target ligand molecule based on the output data.

18. The electronic device according to claim 17, wherein the initial ligand molecule comprises a known ligand molecule that binds to the target protein, and wherein determining, based on the target protein, the decomposition prior distribution of the scaffold and arms in the initial ligand molecule comprises:
obtaining the known ligand molecule that binds to the target protein;
decomposing, based on a binding situation between a plurality of atoms of the known ligand molecule and a binding pocket of the target protein, the plurality of atoms into a plurality of clusters, wherein one cluster among the plurality of clusters serves as the scaffold, and the remaining clusters among the plurality of clusters serve as the arms;
determining, based on coordinates of the atoms in each of the plurality of clusters, the mean matrix and covariance matrix of each of the plurality of clusters; and
determining the decomposition prior distribution based on the mean matrixes and covariance matrixes of the plurality of clusters.

19. A non-transitory computer-readable storage medium, which stores a computer program, wherein the program, when executed by a processor, causes the processor to:
obtain a trained ligand generation model, wherein the trained ligand generation model is generated based on decomposition of a ligand by modeling of atom positions, atom types, and chemical bonds; and
obtain, based on a target protein, a target ligand molecule corresponding to the target protein by using the trained ligand generation model.

20. The non-transitory computer-readable storage medium according to claim 19, wherein obtaining, based on the target protein, the target ligand molecule corresponding to the target protein by using the trained ligand generation model comprises:
determining, based on the target protein, a decomposition prior distribution of a scaffold and arms in an initial ligand molecule, wherein the decomposition prior distribution comprises mean matrixes and covariance matrixes of a plurality of clusters;
determining initial data based on the decomposition prior distribution by sampling, wherein the initial data comprises initial atom positions, initial atom types, and initial chemical bonds;
inputting the initial data to the trained ligand generation model to obtain output data, wherein the output data comprises output atom positions, output atom types, and output chemical bonds; and
generating the target ligand molecule based on the output data.
